(19) United States
(12) Patent Application Publication
(10) Pub. No.: US 2002/0098194 A1
(43) Pub. Date: Jul. 25, 2002

(54) METHODS FOR INDUCING IMMUNITY

(76) Inventors: PAUL V. LEHMANN, CLEVELAND HEIGH, OH (US); THOMAS FORSTHUBER, CLEVELAND, OH (US)

Correspondence Address: KAMRIN T. MACKNIGHT, MEDLEN AND CARROLL, SUITE 2200, 220 MONTGOMERY STREET, SAN FRANCISCO, CA, 94104, US

(21) Appl. No.: 08/621,725

(22) Filed: Mar. 21, 1996

(51) Int. Cl. G01N 33/567; A61K 39/00; A61K 39/38; C12N 5/00; C12N 5/02

(52) U.S. Cl. 424/184.1; 435/325; 435/7.21

(57) ABSTRACT

The present invention relates to methods for inducing immunity, and in particular for inducing immunity that is protective against autoimmunity. In accordance with the present invention, immunity to protein antigens in adult humans is achieved by immunization with Autoimmune Target Antigen (ATA).

### FIELD OF THE INVENTION

The present invention relates to methods for inducing immunity, and in particular for inducing immunity that is protective against autoimmunity.

### BACKGROUND

An “autoimmune” disease is understood to be one where the target of the disease is “self” or “self antigen.” There are a number of diseases that are believed to involve T cell immunity directed to self antigens, including (but not limited to) multiple sclerosis (MS), Type I diabetes, and Rheumatoid arthritis.

Multiple Sclerosis

Multiple sclerosis is a disorder of the central nervous system characterized by zones of demyelination, called plaques, on the myelin sheath. MS can be manifest in deficient motor skills of varying severity, including paralysis. There are also typically symptoms of visual impairment such as blurred vision. Psychiatric disturbances such as memory loss and attention deficit are seen and up to one-half of all patients with the disease require medication to treat major depression.

Most human T cell-mediated autoimmune diseases, including multiple sclerosis, occur spontaneously and are characterized by an insidious onset. See D. E. Smilek et al., Immunol. Rev. 118:37-71 (1990). MS is typically progressive with increasing morbidity. Moreover, men with MS have a significantly reduced life expectancy. See generally, Raine, C. S., 1983, Multiple Sclerosis —Pathology, Diagnosis, and Management, J. Hallpike, C. W. M. Adams, W. W. Tourtelotte (Eds.) (London: Chapman & Hall). Current therapy is unsatisfactory.

The evidence supporting the view that MS is due to immunologic mechanisms comes from histological analysis of MS in humans, as well as work on experimental allergic encephalomyelitis (EAE) in animals. With respect to histological analysis, lesions found in the white matter of patients with multiple sclerosis frequently reveal lymphocyte infiltrates. This underscores the inflammatory cellular immune model for the disease.

Animal studies with EAE provide support for the model. EAE demonstrates significant similarities to multiple sclerosis. See generally, E. Alvord, Experimental Allergic Encephalomyelitis: A Useful Model for Multiple Sclerosis, Progress in Clinical and Biological Research, E. C. Alvord et al. (Eds.) (New York, N.Y.) (1984). EAE is an autoimmune disease mediated by antigen-specific, class II-restricted CD4⁺ T cells. See S. Zamvil and L. Steinman, Ann. Rev. Immunol. 8:579-621(1990). Like MS, EAE is an acute, inflammatory, demyelinating disease with certain forms characterized by relapsing paralysis.

The primary antigen in EAE is myelin basic protein (MBP), a predominant protein on the myelin sheath of the central nervous system. EAE can be induced in susceptible strains of mice by immunization with MBP in Complete Freund's Adjuvant (CFA) accompanied by injections of pertussis toxin. See J. Kimball, Introduction to Immunology (2d Edition) Macmillan Publishing, NY 1986)(p. 488).

Diabetes Mellitus

Type I diabetes is a catabolic disorder in which circulating insulin is virtually absent and where pancreatic cells fail to respond to insulinogenic stimuli. Exogenous insulin is therefore necessary to reduce elevated blood glucose levels.

Evidence supporting the notion that Type I diabetes is due to immunologic mechanisms comes from drug treatment results, as well as work on experimental animal models. With respect to drug treatment, pancreatic cell damage appears to be lessened when immunosuppressive drugs such as cyclosporine are administered. Goodman and Gilman's, The Pharmacological Basis of Therapeutics (8th Edition) (Pergamon Press) (p. 1270). This suggests the importance of autoimmunity as a factor in diabetes pathogenesis.

The role of autoimmunity is supported in animal models. In a diabetic mouse model, a Th1 cell response develops to an epitope in glutamic acid decarboxylase (GAD) at the onset of insulitis. Subsequently, T cell reactivity to other epitopes is observed as well as reactivity to other antigens on islet cells. Induction of T cell tolerance to GAD has been reported to prevent T cell reactivity to these epitopes.

Rheumatoid Arthritis

Rheumatoid arthritis is a chronic inflammatory disorder characterized by joint pain. The course of the disease is variable, but can be both debilitating and mutilating.

Evidence of the autoimmune etiology of the disease come from a variety of sources. First, pregnancy relieves the symptoms of rheumatoid arthritis through a poorly characterized immunosuppression. Second, patients with rheumatoid arthritis who contract the acquired immunodeficiency syndrome (AIDS), and thereby lose significant T cell populations, experience lifelong remissions of the arthritis.

While the antigen targeted by T cells is not known, attention has been given to type II (cartilage) collagen. Indeed, therapeutic trials with this antigen, given orally, have been instituted in the hope of generating tolerance.

### SUMMARY OF THE INVENTION

The present invention relates to methods for selectively inducing immunity, and in particular for inducing immunity that is protective against autoimmunity. The method employs Selective Th Response Inducing Adjuvants (SThRIA). SThRIA are defined functionally as those adjuvants which induce either a Th1 response or a Th2 response upon administration. The present invention also relates to methods of screening for T helper 1 (Th1) Response Inducing Adjuvants (Th1RIA) and T helper 2 (Th2) Response Inducing Adjuvants (Th2RIA).

In accordance with the present invention, immunity to protein antigens in adult humans is achieved by their injection of an Autoimmune Target Antigen (ATA). In one embodiment, the ATA is part of an immunizing preparation comprising Incomplete Freund's Adjuvant (IFA). While it is not intended that the present invention be limited by the mechanism by which protective immunity is achieved, nor to the Th2RIA used, it is believed that the use of IFA induces unipolar T helper 2 (Th2) type immunity (i.e., a Th2RIA), as opposed to the immunization with Complete Freund's Adjuvant (CFA), which triggers unipolar T helper 1 (Th1) type immunity. (CFA is a Th1RIA). These differentially polarizing effects of the adjuvants were not known. The resistance to autoimmune disease that develops following injection of the ATA in IFA is a consequence of immune deviation, not tolerance in the immunological sense.

In one embodiment, the present invention contemplates a method of immunizing an adult human, comprising: a) providing: i) an adult with symptoms of autoimmune disease, and ii) an immunizing preparation comprising an Autoimmune Target Antigen and a Th2 Response Inducing Adjuvant; and b) immunizing said adult with said immunizing preparation under conditions such that said symptoms are reduced.

In another embodiment, the method further comprises: c) obtaining a primary cell population from said immunized adult comprising T cells capable of secreting cytokines; d) adding said primary cell population to said microwell comprising a hydrophobic membrane having a first cytokine binding ligand, under conditions such that said T cell secretes a cytokine that binds to said first cytokine binding ligand; and e) detecting said secreted T cell cytokine.

It is not intended that the present invention be limited by the mode or site of immunization. In one embodiment, the immunizing of said adult is intraperitoneal. Other routes of administration are contemplated, including intramuscular and subcutaneous immunization. It is also not intended that the present invention be limited to treatment of adults. There are autoimmune diseases (e.g., Type 1 Diabetes) where treatment of juveniles may be indicated.

In one embodiment of the method of the present invention, said Th2 Response Inducing Adjuvant is Incomplete Freund's Adjuvant. In one embodiment, said adult has symptoms of multiple sclerosis and said Autoimmune Target Antigen is myelin basic protein. In another embodiment, said adult has symptoms of multiple sclerosis and said Autoimmune Target Antigen is proteolipid protein. In still another embodiment, said adult has symptoms of Type 1 Diabetes and said Autoimmune Target Antigen is glutamic acid decarboxylase. In a further embodiment, said adult has symptoms of Behcet's disease and said Autoimmune Target Antigen is intra photoreceptor binding protein or, alternatively said Autoimmune Target Antigen is S-antigen. In yet another embodiment, said adult has symptoms of interstitial nephritis and said Autoimmune Target Antigen is renal tubular antigen.

The present invention also contemplates improved vaccines. In one embodiment, the present invention contemplates a method of immunizing an adult human, comprising: a) providing: i) an adult with symptoms of infection by a pathogenic organism, and ii) an immunizing preparation comprising one or more antigens of said pathogenic organism in a Selective Th Response Inducing Adjuvant; and b) immunizing said adult with said immunizing preparation under conditions such that said symptoms are reduced. In one embodiment, the method further comprises the steps of: c) obtaining a primary cell population from said immunized adult comprising T cells capable of secreting cytokines; d) adding said primary cell population to said microwell comprising a hydrophobic membrane having a first cytokine binding ligand, under conditions such that said T cell secretes a cytokine that binds to said first cytokine binding ligand; and e) detecting said secreted T cell cytokine.

It is not intended that the present invention be limited by the nature of the pathogen. In one embodiment, said pathogen is an intracellular pathogen. In another embodiment, said pathogen is an extracellular pathogen. It is believed that a Th1RIA will be useful in immunizing against intracellular pathogens, while a Th2RIA will be useful in immunizing against extracellular pathogens. It is also not intended that the present invention be limited to adults, many vaccines have applications in children.

In one embodiment, the extracellular pathogen is a member of the Helicobacter genus. In another embodiment, said pathogen is

H. pylorii

### DESCRIPTION OF THE INVENTION

The present invention relates to methods for selectively inducing immunity, and in particular for inducing immunity that is protective against autoimmunity. While it is not intended that the present invention be limited by the mechanism by which successful protection against autoimmunity is achieved, it is believed that the method of the present invention induces an anti-inflammatory response mediated by Th2 cells. The description of the invention that follows discusses I) Incomplete Freund's Adjuvant, II) Generation of Selective Th Immunity, III) Measuring Selective Th Immunity, and IV) Protection Against Autoimmunity with Autoimmune Target Antigen (ATA). The present invention also relates to V) Improved Vaccines for generating immunity against pathogenic organisms.

I. Incomplete Freund's Adjuvant (IFA)

In the past, adjuvants were believed to function as non-specific immune simulators and are frequently used to enhance the production of antibody. By providing a pool of emulsified antigen at an injection site, adjuvants were thought to allow the antigen to be released slowly over an extended period of time, thereby enhancing and prolonging the immune response. See generally, I. Roitt et al., Immunology (section 8.9) (Gower Medical Publishing 1985).

Equal portions of a diluent such as saline, in which the antigen is dissolved or suspended, and an emulsifier-mineral oil mixture (which may or may not contain mycobacteria) are mixed until a stable water in oil emulsion forms. Emulsification should be thorough and is particularly important if the antigen is a soluble protein. The resulting emulsion is satisfactory if a drop placed on the surface of water will not spread.

The most commonly used adjuvants in animals are Complete Freund's Adjuvant (CFA) and Incomplete Freund's Adjuvant (IFA). CFA is a suspension of Mycobacterium butyricum in a mixture of paraffin oil and emulsifying agent mannide monoleate. IFA is similar to CFA except that M. butyricum has been omitted.

The addition of mycobacteria to these adjuvants (such as in CFA) further enhances the immune response to antigens, particularly soluble antigens. However, these bactoadjuvants are only used in the preparation of antigen-adjuvant emulsions used in immunological studies with laboratory animals; they are not intended for human use or therapeutic use. Accidental human injections with CFA have resulted in severe inflammatory reactions. On the other hand, the present invention contemplates pertussis toxin (without CFA) as a Th1RIA which can be safely administered in humans.

The present invention demonstrates that adjuvants can generate a specific immune response. By using the teachings of the present invention, the nature of the immune response (Th1 or Th2) can be controlled.

I Generation Of Selective Th Immunity

The present invention contemplates generating selective Th1 or Th2 immunity. In general, T cells can act in different subpopulations that utilize strikingly different effector functions. T cell responses can be pro-inflammatory T helper 1 type, Th1, characterized by the secretion of interferon gamma (IFNγ) and interleukin 2 (IL-2). Th1 cells are critical for the cellular defense and provide little help for antibody secretion. (Strong Th1 responses are usually associated with poor antibody production, which highlights the importance of directly measuring the T cell response instead of relying on antibody measurements.) The other class of T cell responses is anti-inflammatory, mediated by Th2 cells that produce IL-4, 5, 10, but no IL-2 or IFNγ. Th2 cells are the helper cells for antibody production. CD4⁺ and CD8⁺ cells both occur in these subpopulations: Th1/Th2:CD4, TC1/TC2:CD8.

Importantly, for each type of infection there is an “appropriate” (and different) type of T cell response (e.g., Th1 vs. Th2, CD4⁺ vs. CD8⁺) that clears the infectious agent but does not cause excessive tissue destruction to the host. It is detrimental to the host if an “inappropriate” type of T cell response is engaged (Th1 instead of Th2, or vice versa). Generally, one would want to induce the Th1 response to clear an intracellular pathogen and a Th2 response to clear an extracellular pathogen.

III. Measuring Selective Th Immunity

The present invention contemplates monitoring and measuring Th1 or Th2 immunity generated according to the method of the present invention. In one embodiment, the present invention contemplates measuring selective Th immunity using an ELISA spot assay. The ELISA spot assay of the present invention involves microplates containing hydrophobic membranes, such as the polyvinylidene difluoride “PVDF” polymer-based membrane available commercially from Gelman Sciences Membrane and Device Division (Ann Arbor, Mich.). Plates containing PVDF membranes are available commercially from Polyfiltronics (Rockland, Mass.). As a membrane, the pore size is generally greater than 0.1 microns and less than 1.0 microns, and preferably approximately 0.45 microns. The PVDF-modified membranes increase the sensitivity of the assay more that 10 fold and dramatically reduce the nonspecific background.

The preferred plate is a plate that is not completely transparent. Transparent plates conduct and refract the light in highly irregular patterns, creating problematic variations in illumination of the membrane. There is also a problem with the highly polished and thereby reflective nature of the interior sides of the wells, which creates distorted mirror images of the spots during analysis, as well as compounding the variations in the lighting.

The preferred plate of the present invention is made from a white, translucent version of the same plastic (not ivory, or any other tint that would introduce color to the light). In selecting the plastic, it is important that it be suitable in tissue culture (i.e., suitable in the sense that the cells do not adhere to the walls, rather than the membrane).

In one embodiment, the present invention contemplates a 96-well microtiter plate comprising a PVDF membrane attached to the perimeter or periphery of each well and enclosed by a solid bottom. This embodiment can be made using a two-plate assembly approach. In this case, the “A plate” has the membrane attached to the periphery of each well. This “A plate” is bonded to a “B plate” which encloses the bottom, i.e., with solid plastic under the membrane. This type of enclosed bottom or “solid bottom” design has been found to have benefits compared to the “open bottom” or “flow through” PVDF membrane-containing plates available from Millipore Corporation (Marlborough, Mass.).

The membrane may be attached to the microwells by any number of conventional technologies including: adhesive attachment, heat sealing, solvent sealing, chemical bonding and ultrasonic welding. Ultrasonic welding is a preferred method of attaching the PVDF membrane to the periphery of the microwells of the present invention. Ultrasonic welding is described in U.S. Pat. Nos. 4,948,442 and 5,047,215 to Roy Manns, hereby incorporated by reference.

In accordance with one embodiment of the invention, the “closed bottom” microwells containing the PVDF membrane are precoated with a first cytokine binding ligand, such as capture antibody specific for the cytokine to be detected (e.g., anti-IFNγmAb1 or anti-IL5 mAb or with both simultaneously for the two color assay). In a preferred method, freshly isolated, primary cell populations (e.g., lymph node, spleen cells, etc.) are subsequently plated with the test antigen, e.g., ATA protein or peptide; control cultures contain irrelevant antigens or peptides. Since the primary cell suspensions contain abundant antigen presenting cells (APC) to process and present the antigen, specific T cells become activated and start to secrete the type of cytokine they are programmed to produce. As the cytokine is released, it is captured around the secreting cells by the plate-bound ligand. After a suitable incubation period (e.g., between 30 minutes and 48 hours) the cell culture is terminated, cells are removed and the plate bound cytokine is visualized by a second cytokine binding ligand (e.g., antibody) free in solution that is conjugated to enzyme. The addition of substrate (not shown) results in an enzymatic color reaction. Each cytokine producing cell will be represented as an ELISA spot.

The assay is not limited by the nature of the T cell product detected. Table 1 (below) provides an illustrative (but not exclusive) list of T cell products detectable by the ELISA spot assay.

IV. Protection Against Autoimmunity

There is a series of human diseases with T cell mediated autoimmune etiology. These include (but are not limited to) multiple sclerosis (MS), rheumatoid arthritis (RA, or primary chronic polyarthritis, PCP), juvenile diabetes (insulin dependent diabetes mellitus, IDDM, Type I diabetes), Hashimoto thyroiditis, Behcet's disease and interstitial nephritis. The present invention contemplates generating protective immunity utilizing the appropriate Autoimmune Target Antigen (ATA). Autoimmune target antigens are those antigens to which the T cell response in vivo is directed for a given autoimmune disease. Table 2 sets forth the major T cell autoimmune diseases known and their associated ATA.

The most common cause of hypothyroidism in the United States is Hashimoto's thyroiditis, an immunologic disorder in genetically predisposed individuals, in which autoimmune destruction of the thyroid occurs. Goiter presents early and is absent in the later stages with the degree of hypothroidism ranging from mild to severe. Generally, the syndrome results from deficiency of thyroid hormones and is manifested largely by a reversible slowing down of all body functions. See B. Katzung, Basic & Clinical Pharmacology (5th Edition) (Appleton & Lange, Norwalk, Conn. 1992)(p. 537).

Evidence to support the autoimmune basis of this disease derives from the animal model of experimental allergic thyroiditis (EAT) in which mice are injected with an aqueous solution of thyroglobulin in Complete Freund's Adjuvant. Thyroid antibodies and a destructive inflammatory lesion in the thyroid reminiscent of that seen in Hoshimoto's thyroiditis is produced.

As noted previously, the primary antigen for multiple sclerosis is myelin basic protein (MBP), a predominant protein on the myelin sheath of the central nervous system. The present invention contemplates inducing protective immunity in adult humans by immunization with MBP in Incomplete Freunds Adjuvant (IFA).

Behcet's disease (also known as intraocular uveoretinitis) is an autoimmune disease for which the present invention also contemplates protective immunity. The animal model for this disease is Experimental Allergic Uveitis (EAU). See R. R. Caspi et al., J. Immunology 140: 1490 (1988). There are two (2) eye antigens that induce the disease, IPRB (intra photoreceptor binding protein) and S-antigen (retinal soluble protein=Arestin).

TABLE 1

| Name | Abbr. | Type | Specific Name |
|---|---|---|---|
| Interferons | IFN | alpha | Leukocyte Interferon |
| | | beta | Fibroblast Interferon |
| | | gamma | Macrophage Activation Factor |
| Interleukins | IL-1 | 1 alpha | Endogenous Pyrogen |
| | | 1 beta | Lymphocyte-Activating Factor |
| | | 1 ra | IL-1 Receptor Antagonist |
| | IL-2 | | T-cell Growth Factor |
| | IL-3 | | Mast Cell Growth Factor |
| | IL-4 | | B-cell Growth Factor |
| | IL-5 | | Eosinophil Differentiation Factor |
| | IL-6 | | Hybridoma Growth Factor |
| | IL-7 | | Lymphopoietin |
| | IL-8 | | Granulocyte Chemotactic Protein |
| | IL-9 | | Megakaryoblast Growth Factor |
| | IL-10 | | Cytokine Synthesis Inhibitor Factor |
| | IL-11 | | Stromal Cell-Derived Cytokine |
| | IL-12 | | Natural Killer Cell Stimulatory |
| | | | Factor |
| Tumor | TNF | alpha | Cachectin |
| Necrosis | | beta | Lymphotoxin |
| Factors | | | |
| Colony | CSF | GM-CSF | Granulocyte-macrophage Colony- |
| Stimulating | | | Stimulating Factor |
| Factors | | Mp-CSF | Macrophage Growth Factor |
| | | G-CSF | Granulocyte Colony-stimulating |
| | | | Factor |
| | | EPO | Erythropoietin |
| Transforming | TGF | beta 1 | Cartilage-inducing Factor |
| Growth | | beta 2 | Epstein-Barr Virus-inducing Factor |
| Factor | | beta 3 | Tissue-derived Growth Factor |
| Other | LIF | | Leukemia Inhibitory Factor |
| Growth | MIF | | Macrophage Migration-inhibiting |
| Factors | | | Factor |
| | MCP | | Monocyte Chemoattractant Protein |
| | EGF | | Epidermal Growth Factor |
| | PDGF | | Platelet-derived Growth Factor |
| | FGF | alpha | Acidic Fibroblast Growth Factor |
| | | beta | Basic Fibroblast Growth Factor |
| | ILGF | | Insulin-like Growth Factor |
| | NGF | | Nerve Growth Factor |
| | BCGF | | B-cell growth factor |

While it is not intended that the present invention be limited by the mechanism by which protective immunity is achieved, based on data on corresponding animal models, all of these disease are believed to be mediated by the pro-inflammatory T cell subset, Th1. Therefore, all of these diseases should be inhibited by the anti-inflammatory Th2 subset.

More generally, the present invention contemplates a method of immunizing an adult human, comprising: a) providing: i) an adult with symptoms of autoimmune disease, and ii) an immunizing preparation comprising an Autoimmune Target Antigen in a Th2 response inducing adjuvant (such as Incomplete Freund's Adjuvant); and b) immunizing said adult with said immunizing preparation under conditions such that said symptoms are reduced.

TABLE 2

| Autoimmune Disease | Autoimmune Target Antigen(s) |
|---|---|
| Multiple sclerosis (MS) | Myelin Basic Protein (MBP), Proteolipid |
| | Protein (PLP) |
| Rheumatoid arthritis (RA) | Type II Collagen |
| Type 1 Diabetes | Glutamic Acid Decarboxylase (GAD) |
| Hashimoto thyroiditis | Thyroglobulin |
| Behcet's disease | Intra photoreceptor binding protein (IPRB), |
| | S-antigen (retinal soluble protein/Arestin) |
| Interstitial nephritis | Renal Tubular Antigen (RTA) |

V. Improved Vaccines

The present invention also contemplates improved vaccines for vaccination against infectious agents. While it is not intended that the present invention be limited by the mechanism by which vaccination is achieved, it is believed that the correct type (Th1 or Th2) of T cell response is critical, if a protective effect rather than a deleterious outcome is to be achieved. The present invention provides the means by which the correct type of response can be determined and monitored. By virtue of the above-described T cell ELISA Spot Assay, the present invention provides methods of screening for T helper 1 (Th1) Response Inducing Adjuvants (Th1RIA) and T helper 2 (Th2) Response Inducing Adjuvants (Th2RIA). To screen, adjuvants are administered and T cells are subsequently tested as set forth above.

It is not intended that the present invention be limited by the particular infectious agent. The present invention contemplates intracellular pathogens such as viruses (including HIV). All extracellular pathogens are contemplated for improved vaccines, including members of the Helicobacter species.

The bacterium now known as Helicobacter pylorii was relatively recently discovered. Reports of the isolation and identification of a highly motile, curved, gram-negative rod was found in human stomach linings and associated with gastritis were published in 1982. B. Marshall, Lancet 1:1273-1275 (1983). Various reports have indicated that the organism has a world-wide distribution. M. J. Blaser, Prin. Pract. Infect. Dis., pp. 3-10 (1991).

There are three members of the new genus, Helicobacter. H. pylorii and H. mustelae were identified first. An additional species, H. felis, was isolated in 1988 and named in 1991. A. Lee et al., Infect. Immun. 56:2843-2850 (12988); B. J. Paster et al., Int'l. J. Sys. Bacteriol. 41:31-38 (1989).

The present invention contemplates an improved vaccine for members of the genus, including vaccines for H. pylorii. In accordance with the present invention, immunity to this species in adult humans is achieved by injection of H. pylorii antigens in Incomplete Freund's Adjuvant (IFA).

Experimental

The following examples serve to illustrate certain preferred embodiments and aspects of the present invention and are not to be construed as limiting the scope thereof.

In the experimental disclosure which follows, the following abbreviations apply: eq (equivalents); M (Molar); μ M (micromolar); N (Normal); mol (moles); mmol (millimoles); μ mol (micromoles); nmol (nanomoles); gm (grams); mg (milligrams); μ g (micrograms); L (liters); ml (milliliters); μ l (microliters); cm (centimeters); mm (millimeters); μ m (micrometers); nm (nanometers); ° C. (degrees Centigrade).

### EXAMPLE 1

As noted above, the animal model for MS in humans is experimental allergic encephalomyelitis (EAE). EAE demonstrates significant similarities to multiple sclerosis. Like MS, EAE is an acute, inflammatory, demyelinating disease with certain forms characterized by relapsing paralysis. The primary antigen in EAE is myelin basic protein (MBP).

In this example, the method of the present invention is shown to provide protection against EAE in mice. Specifically, six to eight week old female mice were given a primary intraperitoneal (“i.p.”) injection of MBP (100 μg of antigen per mouse) in IFA (or a control antigen in IFA). Subsequently, MBP is reinjected in a subcutaneous (“s.c.”) secondary injection using CFA along with pertussis (PTX) as required normally for EAE induction.

Table 3 shows that preinjection with the CNS proteins MBP or PLP, in IFA, protects from subsequent EAE development (clinical disease >grade 2, i.e., ≧hind leg paralysis). The injection of an irrelevant control antigen, hen egg white lysozyme (HEL), in IFA has no protective effect; that is to say, injection of MBP in CFA with pertussis (0.1 μg, i.v., O and 48 h after immunization) in control-primed animals resulted in EAE induction.

TABLE 3

| | Injection With Antigen (ATA) | | |
|---|---|---|---|
| Mice | Primary | Secondary | Results (EAE) |
| B10.PL | MBP/IFA, i.p. | MBP/CFA, s.c., PTX | 0/8 |
| B10.PL | HEL/IFA, i.p. | MBP/CFA, s.c., PTX | 7/8 |
| SJL | PLP/IFA, i.p. | PLP/CFA, s.c., PTX | 0/11 |
| SJL | HEL/IFA, i.p. | PLP/CFA, s.c., PTX | 8/11 |

### EXAMPLE 2

As noted above, the present invention contemplates monitoring and measuring Th2 immunity generated according to the method of the present invention. In one embodiment, the present invention contemplates measuring Th2 immunity using a T Cell ELISA Spot Assay. In this example, cytokines were measured by the Assay three weeks after injection. The results are shown in Table 4; the data are expressed as the arithmetic mean of 8-10 spleens tested individually. The numbers of spots generated equal the number of antigen specific T cells in the million spleen cells tested for each mouse.

TABLE 4

| | | Cytokine Response To Antigen | | | | | |
|---|---|---|---|---|---|---|---|
| | Injection | MBP | | HEL | | PLP | |
| Strain | Antigen/Adjuvent | IFNγ | IL-5 | IFNγ | IL-5 | IFNγ | IL-5 |
| B10.PL | MBP/IFA/i.p. | <5 | 66 | <5 | <5 | <5 | <5 |
| B10.PL | MBP/CFA/s.c. | 83 | <5 | <5 | <5 | <5 | <5 |
| B10.PL | HEL/IFA/i.p. | <5 | <5 | <5 | 34 | <5 | <5 |
| B10.PL | HEL/CFA/s.c. | <5 | <5 | 59 | <5 | <5 | <5 |
| SJL | PLP/IFA/i.p. | <5 | <5 | <5 | <5 | <5 | 82 |
| SJL | PLP/CFA/s.c. | <5 | <5 | <5 | <5 | 63 | <5 |
| SJL | HEL/IFA/i.p. | <5 | <5 | <5 | 40 | <5 | <5 |
| SJL | HEL/CFA/s.c. | <5 | <5 | 47 | <5 | <5 | <5 |

While it is not intended that the present invention be limited by the mechanism by which protection against autoimmunity was achieved in Example 1, it is believed from such data as shown in Table 4 that the primary injection of MBP in IFA induces Th2 immunity in naive animals. On the other hand, injections with MBP in CFA (with pertussis antigen) induces Th1 immunity in naive animals. However, it is believed that when the antigen is reinjected in CFA into IFA primed mice, the response retains its original Th2 character.

### EXAMPLE 3

The results of Example 1 show that preinjection with the CNS proteins MBP or PLP, in IFA, protects from subsequent EAE development. In this Example, six to eight week old female mice were given a primary intraperitoneal (“i.p.”) injection of MBP (100 μg of antigen per mouse) in IFA. Subsequently, MBP is reinjected i.p. in IFA with pertussis (0.1 μg, i.v., O and 48 h after immunization). A T Cell ELI SA Spot Assay was used to measure cytokines three weeks after injection. The results are shown in Table 5; the data are expressed as the arithmetic mean of 8-10 spleens tested individually. The numbers of spots generated equal the number of antigen specific T cells in the million spleen cells tested for each mouse.

Challenge with PTX was found to be associated with EAE development. The results in Table 5 indicates that pertussis toxin (PTX) converts the IFA induced Th2 response into a Th1 response. Thus, PTX is a Th1 response inducing adjuvant.

TABLE 5

| Immunization | | MBP Recall Response | | |
|---|---|---|---|---|
| Mice | Injected | IL-5 | IFNγ | EAE |
| B10.PL | MBP/IFA, i.p. | 61 | <5 | 0/10 |
| B10.PL | MBP/IFA, i.p. + PTX | 8 | 73 | 6/10 |

### EXAMPLE 4

In this example, the method of the present invention is shown to provide protective immunity for interstitial nephritis. Using the T Cell ELISA Spot Assay, it was found that RTA (renal tubular antigen, the autoantigen that induces interstitial nephritis) induces Th2 immunity when injected in IFA (results not shown). On the other hand, RTA induces Th1 immunity when injected in CFA (results not shown).

When the method of the present invention was tested in mice, it was found that RTA/IFA does not induce interstitial nephritis in SJL mice (no infiltrating lymphocytes were observed in the tubules). In contrast, lymphocytic infiltrates were seen in tubules of an RTA/CFA immunized mouse (data not shown). RTA/IFA pre-injected mice do not develop infiltrates when reinjected with RTA/CFA (data not shown). These results indicate that the rules established for EAE (see above) also apply for the autoimmune model of interstitial nephritis.

From the above, it should be clear that the present invention provides methods to selectively induce immunity, and in particular to induce immunity that is protective against autoimmunity.

1. A method of immunizing an adult human, comprising:
a) providing: i) an adult with symptoms of autoimmune disease, and ii) an immunizing preparation comprising an Autoimmune Target Antigen and a Th2 Response Inducing Adjuvant; and
b) immunizing said adult with said immunizing preparation under conditions such that said symptoms are reduced.

2. The method of claim 1, further comprising the steps of:
c) obtaining a primary cell population from said immunized adult comprising T cells capable of secreting cytokines;
d) adding said primary cell population to said microwell comprising a hydrophobic membrane having a first cytokine binding ligand, under conditions such that said T cell secretes a cytokine that binds to said first cytokine binding ligand; and
e) detecting said secreted T cell cytokine.

3. The method of claim 1, wherein said adult has symptoms of multiple sclerosis and said Autoimmune Target Antigen is myelin basic protein.

4. The method of claim 1, wherein said adult has symptoms of multiple sclerosis and said Autoimmune Target Antigen is proteolipid protein.

5. The method of claim 1, wherein said adult has symptoms of Type 1 Diabetes and said Autoimmune Target Antigen is glutamic acid decarboxylase.

6. The method of claim 1, wherein said adult has symptoms of Behcet's disease and said Autoimmune Target Antigen is intra photoreceptor binding protein.

7. The method of claim 1, wherein said adult has symptoms of Behcet's disease and said Autoimmune Target Antigen is S-antigen.

8. The method of claim 1, wherein said adult has symptoms of interstitial nephritis and said Autoimmune Target Antigen is renal tubular antigen.

9. The method of claim 1, wherein said Th2 Response Inducing Adjuvant is Incomplete Freund'Adjuvant.

10. A method of immunizing an adult human, comprising:
a) providing: i) an adult with symptoms of infection by a pathogenic organism, and ii) an immunizing preparation comprising one or more antigens of said pathogenic organism and a Selective Th Response Inducing Adjuvant; and
b) immunizing said adult with said immunizing preparation under conditions such that said symptoms are reduced.

11. The method of claim 10, further comprising the steps of:
c) obtaining a primary cell population from said immunized adult comprising T cells capable of secreting cytokines;
d) adding said primary cell population to said microwell comprising a hydrophobic membrane having a first cytokine binding ligand, under conditions such that said T cell secretes a cytokine that binds to said first cytokine binding ligand; and
e) detecting said secreted T cell cytokine.

12. The method of claim 10, wherein said pathogen is an intracellular pathogen.

13. The method of claim 10, wherein said pathogen is an extracellular pathogen.

14. The method of claim 10, wherein said Selective Th Response Inducing Adjuvant is Incomplete Freund's Adjuvant.

15. The method of claim 10, wherein said Selective Th Response Inducing Adjuvant is Pertussis toxin.

16. Th method of claim 13, wherein said pathogen is a member of the Helicobacter genus.

17. The method of claim 16, wherein said pathogen is H. pylorii.
